(19) United States
(12) Patent Application Publication
(10) Pub. No.: US 2009/0018532 A1
(43) Pub. Date: Jan. 15, 2009

(54) LASER FEMTOSECOND MICROTOME FOR CUTTING OUT A MATERIAL SLICE BY A LASER BEAM, IN PARTICULAR IN A CORNEA

(75) Inventor: Francois Salin, Gradignan (FR)

Correspondence Address: YOUNG & THOMPSON, 209 Madison Street, Suite 500, ALEXANDRIA, VA, 22314, US

(73) Assignee: CENTRE NATIONAL DE LA RECHERCHE SCIENTIFIQUE, Paris (FR)

(21) Appl. No.: 12/092,621

(22) PCT Filed: Nov. 3, 2006
(86) PCT No.: PCT/FR06/51136

§ 371 Date: Sep. 11, 2008

(30) Foreign Application Priority Data
Nov. 4, 2005 (FR) 0553345

(51) Int. Cl. A61F 9/008; A61B 18/20

(52) U.S. Cl. 606/5

(57) ABSTRACT

A laser femtosecond microtome for cutting out by a focused laser beam at least one slice of material in a material block, wherein the block includes a front surface and the slice carrying the front surface, the slice extends at least partially substantially in a X, Z plane perpendicular to an axis Y of the block thickness, the slice is separated from the remaining part of the block by a cleavage surface formed by an assembly of bubbles brought together, each bubble is formed in a focus area of at least one convergent laser beam pulse of an optical axis L. According to the invention, the optical axis L of the convergent part (3) of the laser beam forms an angle ranging between −45° and ±45° relative to the X, Z. The ellipsoid-shaped focus area has its smaller axis in the direction of the axis Y.

The invention relates to a laser femtosecond microtome intended for cutting out in a material block a slice of material thanks to a focused laser beam. The material may be the cornea of an eye or any other material wherein it is possible to obtain a cleavage by forming bubbles in focus areas of the laser beam as for instance in certain plastic materials. It finds application in particular in the field of micro-machining of parts, for example optical parts, or in the field of processing the visual defects of the eye. In the latter application, it enables in particular to create a cavity in the eye which is compatible with intra-stromal surgery, corneal surgery and myopia, hypermetropia or astigmatism correction.

A large portion of the world population suffers from visual defects. Most these defects are due to a deformation of the eye which is not perfectly spherical any longer. Spectacles or contact lenses are used currently to correct such defects. For some years it has been possible to correct some of such vision defects by sculpting the cornea directly using a laser. This technique requires first of all opening a lid at the surface of the cornea so as to enable then the action of an ultraviolet laser which enables to correct the shape of the cornea. Finally, it is contemplated, in the long run, to correct short-sightedness directly without opening any lids.

Several methods are available for opening such a lid. The first uses a mechanical microtome with a blade for cutting out a thin slice of the cornea. In this method the microtome is called a metallic microkeratome. It enables to provide regular surface condition which the ultraviolet laser may work. The metallic microkeratome exhibits however the shortcoming of requiring a material contact with the cornea and hence a risk of infection. Moreover there remains a significant proportion of failed cutting-outs due to variations in dimensional parameters of the cornea from one patient to another.

The second, called femtolaser microkeratome, uses a femtosecond laser in the axis of the eye for cutting out a slice of cornea but the surface condition obtained is not satisfactory since the cleavage area obtained by forming bubbles within the cornea is relative thick and irregular and the result is a stamp-shaped tear at the interface between the lid and the cornea. However, the femtosecond laser exhibits the advantage of not introducing any risk of infection since there is normally no material contact with the cornea during the laser cutting-out operation. In practice, until now, metallic microkeratome is preferably used for obtaining satisfactory results.

Thus the method called LASIK (LAser In Situ Keratomyleusis) for correcting short-sightedness which consists in modifying the curvature of the cornea by laser ablation is the refractive surgical intervention most frequently practiced in 2002. The first step of a LASIK is the cutting-out of a slice of cornea in the form of a superficial lid using a razor blade of a metallic microkeratome. This lid, which must exhibit a thickness of approximately 150 μm and a diameter of 7 to 9 mm, remains attached to the surface by a tissue hinge left out when cutting out. In a second step, the lid is reclined long enough to perform a surface ablation using an ultraviolet excimer laser. This laser emits a 193 mm ultraviolet radiation highly absorbed at the surface of the cornea which is then volatilised. The corneal curvature is thus remodeled by selective and internal thinning of the corneal stroma. Once the intervention is completed, the lid is simply put back in place.

The femtolaser microkeratome laser uses a femtosecond laser beam which is focused with a focus approximately 150 μm below the surface of the cornea and possesses an optical axis substantially parallel to that of the eye and hence corresponds to frontal application of the laser beam to the cornea of the eye. The high intensity generated at the focal point produces a bubble of vaporised material which causes local disruption of the cornea. By moving the focal point laterally a carpet of jointing bubbles forming a cleavage area within the cornea can be created. The lid is cut out by laser while conducting a sagittal cut-out from the bubble plane up to the surface. Although this method does work, it exhibits the shortcoming of causing a cleavage area which is far less clearly defined than with a metallic microkeratome and leaves a surface roughness which is detrimental to healing. The reason is a localisation and an imperfect shape of the bubbles resulting from the anisotropic spatial distribution of the energy deposition of a laser beam around its focusing point.

Indeed, by nature a laser beam is focused with an energy deposition law which is largely anisotropic. It is possible for instance to assess at the focal point the shape of the volume composed of the spots whereof the illumination is greater than half the maximum illumination in the case of a Gaussian circular incident beam and of a focusing means fulfilling an isotropic transfer function. The transversal dimension of this volume is in relation with the waist w₀ (1/e² radius) and is of the order of 1.18 w₀ for the spots halfway up the maximum illumination. The longitudinal dimension of this volume, i.e. in the direction of the focused beam, is given by the Rayleigh length: zᵣ=πw²₀/λ with λ=λ₀/n. It can be noticed whereas this volume is an ellipsoid which is of revolution in the case of a symmetrical incident beam and an isotropic focusing means. The ratio between the smaller axis and the greater axis is: zᵣ/d₀=πw₀/1.18λ. It can be seen that if w₀ is vastly greater than λ, the ellipsoid will be very elongated longitudinally. For instance for a medium of index n=1.3 and w₀=5 μm, a ratio of the order of 18 is obtained. It is hence very difficult to obtain good longitudinal resolution, i.e. in the direction of the optical axis of the incident beam and the cleavage area is hence very high and very irregular.

The solution for obtaining small-sized longitudinal bubbles consists in using very open optical which make the system complicated and costlier and do not allows fields compatible with the LASIK application.

The present invention, instead of trying to correct this defect in connection with the existence of an energy deposition along an ellipsoid-shaped anisotropic isoenergetic (=iso-luminous) distribution, uses it on the contrary for facilitating and improving the quality of the cut-out. Indeed, if the eye is illuminated sideways with the laser, i.e. laterally and not along the optical axis of the eye any longer as previously, the height of the cleavage area is given by the smaller axis of the ellipsoid i.e. a few microns whereas the field depth, into the longitudinal direction corresponding to the general cutting-out plane, i.e. the greater axis of the ellipsoid, facilitates the realisation of the cleavage area. Thus for optical apertures of the order of 0.3 to 0.8, the height of the cleavage area which corresponds to the smaller axis, of transversal direction, may be smaller than a micron.

The invention hence benefit from the ellipsoid shape of the focus area which corresponds substantially to the shape of the bubble created by a laser pulse for, on the one hand, exhibiting the smaller axis of the ellipsoid which determines the height of the cleavage area (hence good accuracy) and the greater axis of the ellipsoid which is in the general plane of the cleavage area (hence faster cleavage, wherein the bubbles extend largely in said plane of the cleavage area).

Thus, in the microtome of the invention, the optical axis of the laser beam converging towards the focus area is arranged substantially laterally relative to the slice of material to provide contrary to the conventional devices whereof the optical axis of the laser beam touches the slice of material perpendicularly. The term slice designates an extended element, planar or not, and with relatively small thickness, even or not, as the case may be.

Thus, the invention relates to a laser femtosecond microtome for cutting out by a focused laser beam at least one slice of material in a material block, wherein the block comprises a front surface and the slice carrying said front surface, the slice extending at least partially substantially in a X, Z plane perpendicular to an axis Y of the block thickness, the slice being separated from the remaining part of the block by a cleavage surface formed by an assembly of bubbles brought together, each bubble being formed in a focus area of at least one convergent laser beam pulse of optical axis L.

According to the invention, the optical axis L of the convergent part of the laser beam forms an angle ranging between −45° and +45° relative to the X, Z plane.

In various embodiments of the invention, the following means which may be combined in all the technically viable possibilities, are employed:

the optical axis L of the beam forms an angle ranging between 10° and +10° relative to the X, Z plane and, preferably, the optical axis L of the beam is substantially in the X, Z plane,

the focused laser beam is obtained by focusing an incident laser beam having a defined transversal illumination section by a focusing means,

the transversal illumination section is selected among the circular or elliptical shapes,

the transversal illumination section is circular,

the transversal illumination section is not circular,

the focusing means includes at least one lens,

the focusing means is a lens,

the focusing means is a set of lenses,

the optical transfer function of the focusing means is isotropic or anisotropic,

the focusing means includes a dynamically addressable wavefront correction system,

the correction system includes a means selected among a deformable mirror, a mosaic of micromirrors or an optical valve with liquid crystals,

the focus area exhibits an isoenergetic distribution of ellipsoid-shaped bubbles, the smaller dimension of said ellipsoid being in a direction substantially parallel the axis Y,

the ratio between the greater axis and the smaller axis of the ellipsoid is above 2 and, preferably greater than 10,

the block of material is the cornea of an eye, the axis Y corresponds substantially to the optical axis of the eye,

an adaptation part made of a material of optical index substantially equal to that of the cornea is arranged on and matches at least the front surface of the cornea, said part having an input face for the convergent beam so that said convergent beam runs through elements having substantially the same optical index,

the input face of the adaptation part is planar and is such as the axis L of the convergent beam is substantially perpendicular to said input face,

the adaptation part compresses and deforms at least the cornea,

the space between the input face of the adaptation part and the focusing means is, in whole or in part, filled with a fluid of index substantially equal to that of the adaptation part or of the focusing means,

the focus area is movable along at least both axes X, Z by computer-controlled actuators,

the focus area is movable along the three axes X, Y, Z by computer-controlled actuators,

the microtome includes moreover a priori localisation means along at least one axis, from the possible position of the bubble by detecting a focusing spot of a light beam not causing any bubbles,

the microtome includes moreover a posteriori localisation means along at least one axis, from the position of the bubble by detecting light of the bubble plasma.

The invention hence enables when applied to corneal surgery the realisation of very thin (reduced in height) microcavities in the eye and hence the realisation of a very thin, and therefore very accurate, cleavage area, by using a focused laser beam whereof the optical axis is quite remote from the optical axis of the eye, the angle between both axes being greater than 45°. Lids for LASIK treatment can thus be provided thanks to a cutting-out operation using a focused laser beam laterally to the cornea.

The quality and the accuracy of the cut-out enable to come closer to the anterior surface of the cornea and to generate lids whereof the thickness may be smaller than 100 μm.

The invention also gives the possibility of performing myopia correction without any incision in the eye simply by realising macrocavities by adding bubbles in the cornea. This type of treatment is also called intra-stromal correction of myopia. Indeed, the femtosecond laser has been suggested to intra-stromal LASIK so as to cut-out inside the cornea a cavity whose collapse causes the variation in curvature of the eye but the lack of accuracy of conventional frontal means with an optical axis parallel to the optical axis of the eye does not enable to provide any accurate correction.

Corneal cut-outs may also be provided for inserting implants in the cornea. Localised cut-outs may also be contemplated for correcting residual optical aberrations.

The invention also enables micro-machining of transparent materials, in particular for the realisation of optical components or applied to microfluidics or micromechanics.

The invention relates finally to an adaptation part for the microtome according to one or several of the previous features and which is made of a plastic material of optical index substantially equal to that of the cornea and of single-use type. The adaptation part may also include one or several of the features listed previously pertaining thereto.

This invention will now be exemplified without being limited thereto with the following description in relation with:

FIG. 1 which represents diagrammatically the convergence of a laser beam in a referential X, Y, Z,

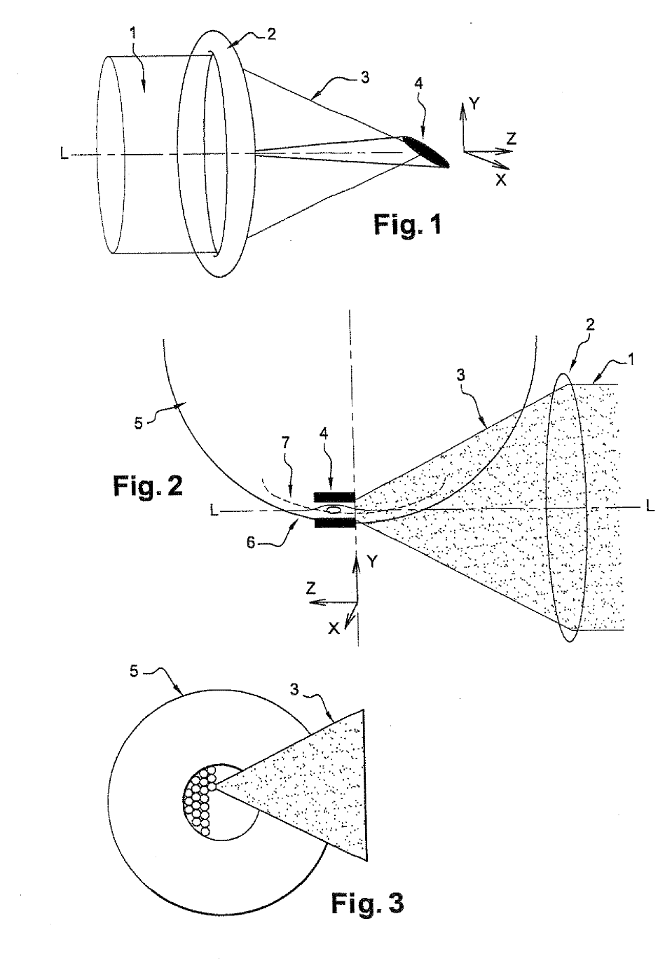

FIG. 2 which represents diagrammatically a sectional lateral view of the process for cutting out a lid of material on the cornea of an eye,

FIG. 3 which represents diagrammatically a frontal lateral view of the process for cutting out a lid of material on the cornea of an eye,

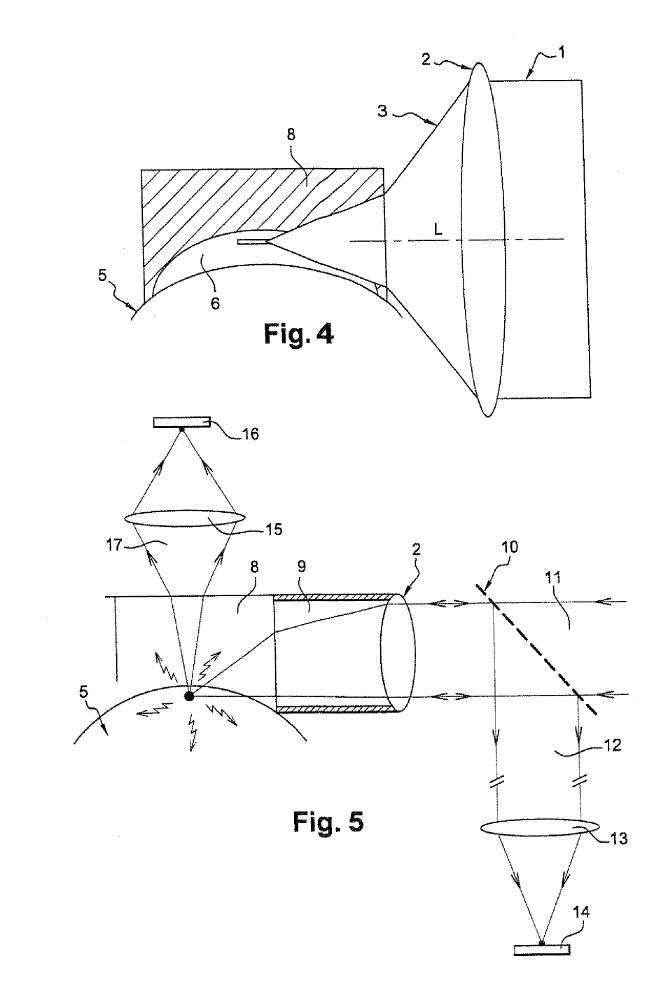

FIG. 4 which represents diagrammatically a sectional lateral view of a variation of the process for cutting out a lid of material on the cornea of an eye,

FIG. 5 which represents diagrammatically the implementation of the invention with means enabling a posteriori back-control of the position of the bubble created.

Most implementation examples of the invention given below relate to the application to the treatment of visual defects of an eye by realising a lid resulting from a cleavage area in the cornea of an eye. More generally, this cleavage area may be more or less high according to the application, in particular of great height by piling up bubbles when realising a macro-cavity and in particular of small height by providing a single layer of bubbles when cutting our a lid as in the following examples. As an alternative to the provision of a macro-cavity, it is possible to cut out a core (two layers of bubbles separated by the core of corneal material) which will then be expelled from the eye by an incision. The shape of the core may be lenticular (biconvex lens) for myopia correction or a biconcave lens for hypermetropia correction, revolution or not (for correcting astigmatism). Similarly, since the example relate to a substantially hemispherical ocular globe, the general shape of the cleavage area matches the general shape of the cornea in particular because when cutting out a circular lid, the latter exhibits substantially constant thickness. However and more generally, for instance when micro-machining another type of object, the shape of the cleavage area will not always be hemispherical but may be planar or exhibit other types of shapes and when realising a slice (detachable or not from the object) its thickness may be constant or not.

It has been seen that the focus area is already ellipsoid with an incident Gaussian beam. The eccentricity of the ellipsoid may still be accentuated or other shapes of focus areas and hence of bubbles may be created by using other illumination shapes of incident laser beam. Thus, a laser beam whereof the transversal geometry is not circular may be used. For instance, by using an elliptical incident laser beam on the focusing means a focal spot is obtained whereof the dimension is still very small in the direction of the optical axis of the eye (axis Y) and large in the other directions. Small-sized bubbles can thus be generated, of a few microns, in a direction parallel to the optical axis of the eye (along Y) and large in both other directions (along X and Z). The time necessary to cutting out a disk-shaped lid can thus be noticeably reduced. It should be understood that in addition to the illumination shape of the incident beam hitting the focusing means, a particular spatial transfer function of said focusing means may on its own or in combination with the illumination shape of the incident beam also enable to spread the focusing area in a plane corresponding to the general plane of the cleavage area and to narrow said focusing are in a plane perpendicular to the cleavage plane.

Arriving from the left section of FIG. 1, an incident laser beam 1 represented schematically as substantially elliptical runs through a focusing means 2, for instance a dioptre or a lens with an isotropic spatial transfer function, enabling to focus it toward a focus area corresponding to the focal point 4 of the optical element. Between the optical element 2 and the focal point 4 the laser beam is converge 3 and of an optical axis L. In the focus area, the iso-illumination distribution curve (or iso-energy) for a given illumination level, corresponding for instance to the threshold illumination level enabling the creation of a bubble (for instance breakdown threshold of the material), has substantially ellipsoid shape whereof the greater axis is substantially in a Z, X plane and the smaller axis substantially parallel to the axis Y of three-dimensional referential X, Y, Z. It can also be noted that the optical axis L of the convergent laser beam 3 is also substantially in the Z, X plane on FIG. 1.

It should be understood that a laser pulse in the material will generate a bubble whereof the shape will now be close to an ellipsoid whereof the greater axis will be in the Z, X plane. It should also be understood that when realising a lid on a cornea the slice of material forming the lid is substantially in a plane parallel to the Z, X plane and that the cleavage area has the smallest possible height along of the axis Y. Thus, not only the accuracy of the cut-out is obtained by the small height of the bubble corresponding to the smaller axis of the ellipsoid, but the cut-out efficiency is increased by the significant length of the bubble corresponding to the greater axis of the ellipsoid in the cleavage plane.

Thus and as applied to the cornea 6 of an eye 5 and represented on FIG. 2, the axis Y is substantially parallel to the optical axis of the eye and the Z, X plane is substantially parallel to at least one portion of the cleavage area and of the lid so that the cleavage area is as little high as possible (corresponding to the smaller axis of the ellipsoid).

On FIG. 2, for simplification purposes, the refraction effects have not been taken into account since a portion of the convergent laser beam 3 runs through a portion of the cornea 6 before reaching the zone of the focal point 4. However, in order to limit or avoid such effects, two solutions may be implemented, the first consisting in tilting the optical axis L relative to the Z, X plane and the second by implementing an optical adaptation part 8 as will be explained in relation with FIG. 4.

To obtain an extended cleavage area, the focus area is moved gradually to provide a two-dimensional matrix unit, lines x columns of bubbles (if requested to provide a lid for the LASIK application) or a three-dimensional matrix unit to realise a macro-cavity (application to in-situ treatment of myopia for instance).

The displacement/trajectory of the focus area to provide this matrix unit of bubbles takes place preferably by starting with the realisation of bubbles in the remotest portion from the laser source and by coming gradually closer so that preferably the convergent beam runs through a corneal portion which has not been cleaved yet. Thus bubbles may be realised by first sweeping along the axis X for a given position Z but away from the source, then reducing the distance on Z by a given pitch and sweeping again along the axis X, and repeating the operation iteratively while reducing gradually the distance on Z as represented diagrammatically on FIG. 3. If a macro-cavity is realised, several sweepings will be made at different positions along the axis Y before decrementing the distance along Z. Other sweeping possibilities are possible but they are such that a portion of the convergent beam runs through a portion of the cornea already comprising bubbles such as for instance a sweeping along Z by starting each time away from the source and by using an incremental displacement along the axis X.

It should be understood that when considering a cornea 6 which is a curved body, the spots will be provided on a surface to suit the needs and for instance to generate a lid of substantially constant thickness of approximately 150 μm the cleavage area 7 must substantially match the shape of the external surface of the cornea at least in its central portion. The focus area is hence then situated approximately 150 μm below the surface of the cornea. The position in Z is set by the position of the lens. The cover which is circular may have a diameter of 9 mm for instance. In the case of a lid which must be folded back, the cut-out should be completed by a circular movement accompanied by a displacement along the axis Y for cutting out the edges of the lid.

More generally, for cutting out a lid, the focus area may be given a trajectory matching the curvature of the cornea which may, in a variation, be flattened optionally using an adaptation part 8 whereof the optical index is close or equal to that of the cornea as will be seen in relation with FIG. 4.

For the implementation of the invention, the position of the focal point is modified using a device enabling electronic and computer-controlled displacement of the lens, more generally the focusing means or any other optical means placed on the path of the beam and able to act on the position of the focal point, at least along the axis Z and, preferably along all axes so as to be able to move the focus area throughout the space X, Y, Z. A priori automatic back-control system of the position of the focus area may also be implemented, either with an additional illumination implemented in the path of the laser beam or when the power of the laser may be reduced at lower level to the creation of bubbles and an optical measuring apparatus on the eye detects the position of the focus area (focal point) thus illuminated by the additional illumination or the laser with small power and enable comparison with an expected position and the generation of a possible correction signal towards the device moving the position of the focal point. The additional illumination may be a LED or another laser but whose power does not enable to create any bubbles. Once the correct position of the focus area reached, the femtosecond laser is activated for one or several light pulses creating the bubble. Independently from an automatic back-control system, the use of an additional illumination may enable the operator to see where the focus area is situated. The possible difference between the wavelengths of the laser and of the additional illumination must be taken into account and the optics implemented must enable the same coincidence of the focal point for both wavelengths or the computer means should take it into consideration.

The back-control may also take place a posteriori by detecting the position of the plasma created when generating a bubble along at least the axis Z and, preferably along the three axes as represented on FIG. 5 which will be explained at a later stage.

FIG. 4 represents a variation of the invention implementing an optical adaptation part 8 which is made of a material of optical index substantially equal to that of the cornea, i.e. an index by approximately 1.33. This part 8 is for instance made of plastic material optionally single-use since in contact with the eye 5. This part 8 is arranged on the anterior portion of the eye and matches at least the front surface of the cornea. The part has a lateral planar input face for the convergent beam 3 such as the axis L of said convergent beam for its own part is substantially perpendicular. Thus, the convergent beam runs through elements, part and cornea, having substantially the same optical index which avoids or limit the refraction effects. The part 8 is preferably interconnected with the equipment comprising the focusing means 2 which has an optical index by approximately 1.55 so as to be able to keep stable dimensional and structural relations between all the optical elements. The focusing means is here represented away from the input face of the adaptation part 8, an air space is provided between both or, in a variation, a space filled with a gel or with an optical adaptation liquid. In a variation not represented, the adaptation part 8 comprises the focusing means, wherein the input face of said part 8 is shaped for focusing the laser beam.

In certain cases a part 8 may be used which, moreover, compresses and deforms at least the cornea of the eye. In a variation, the input face of the adaptation part which is planar may be tilted relative to the optical axis L of the convergent beam 3. In a variation, the input face of the adaptation part is not planar but exhibits a curvature so as to modify the convergence of the convergent beam 3.

It should be noted the focusing means 2 may include more than one lens, in particular to improve the features of the focal point and guarantee small extension along the axis Y in the whole XZ plane.

External means for modifying the wavefront may be inserted on the path of the beam 11 so as to correct the geometrical aberrations of the focusing means 2. These means for modifying the wavefront may be in particular a deformable mirror, a mosaic of micro-mirrors or a liquid crystal optical valve. These means for modifying the wavefront are then actuated dynamically in relation with the position of the focal spot in the field of the lens by a computer means in relation to information stored in advance on the geometrical aberrations of said focusing means 2.

The additional means represented on FIG. 5 enable a posteriori location of the position of the bubble which has just been created by detecting the light waves of the corresponding plasma. An adaptation part 8 is arranged on the cornea and the laser beam 11 arrives laterally through a blade 10, the focusing means 2, a space 9 optionally filled with a fluid (a gel in particular) for optical adaptation and the lateral input face of the part 8. The focusing means 2 is held in relatively stable position relative to the adaptation part 8 by spacers and/or by an encapsulation also enabling to confine the fluid in the space 9. The light waves of the plasma are on the one hand detected forwardly by a beam 17 focused at 15 toward a first, preferably matrix, detector 16 on two dimensions and at least 2×2. The light waves of the plasma are also detected by a second detector 14 preferably also of matrix type on two dimensions and at least 2×2, wherein the corresponding beam 12 has been returned by the blade 10 and focused 13 on the second detector 14. One or both detectors may be implemented. As the first detector 16 may be sufficient on its own for detecting the bubble position in the three dimensions, the matrix sensor provides with two dimensions and an adjusting means for tuning the image focused on the detector indicates the depth. For the second detector 14, according to the same principle the position of the bubble may be obtained in the three dimensions but the axes given by the matrix sensor will be different relative to the first case since the observation is lateral and not frontal any longer.

It should be understood that these examples are purely illustrative and that the invention may be offered in various other embodiments obvious to the man of the art, without the latter having to demonstrate any inventiveness and without departing from the general framework of the invention such as delineated by the claims.

1-14. (canceled)

15. A laser femtosecond microtome for cutting out by a focused laser beam by a focusing means (2) of at least one slice of material in a block of material, wherein the block comprises a front surface and the slice carrying said front surface, the slice extending at least partially substantially on a X, Z plane perpendicular to an axis Y of the block thickness, the slice being separated from the remaining part of the block by a cleavage surface formed by an assembly of bubbles brought together, each bubble being formed in a focus area of at least one convergent laser beam pulse of optical axis L, characterised in that it includes means so that the optical axis L of the convergent part (3) of the laser beam arrives substantially laterally relative to the block by forming an angle ranging between −45° and +45° with respect to the plane X, Z plane.

16. A microtome according to claim 15, characterised in that said means enable the optical axis of the beam to form an angle ranging between −10° and +10° relative to the X, Z plane and, preferably, so that the optical axis L of the beam is substantially in the X, Z plane.

17. A microtome according to claim 15, characterised in that the focusing means (2) includes at least one lens.

18. A microtome according to claim 15, characterised in that it includes means so that the focus area exhibits an isoenergetic distribution of ellipsoid-shaped bubbles, the smaller dimension of said ellipsoid being in a direction substantially parallel the axis Y.

19. A microtome according to claim 18, characterised in that it comprises means so that the ratio between the greater axis and the smaller axis of the ellipsoid is above 2 and, preferably greater than 10.

20. A microtome according to claim 18, characterised in that the focusing means fulfils anisotropic spatial transfer function and the isoenergetic distribution is obtained by means of the microtome producing an incident laser beam with a defined transversal illumination section which is focused by the focusing means.

21. A microtome according to claim 19, characterised in that the focusing means fulfils anisotropic spatial transfer function and the isoenergetic distribution is obtained by means of the microtome producing an incident laser beam with a defined transversal illumination section which is focused by the focusing means.

22. A microtome according to claim 15, characterised in that the focusing means includes a dynamically addressable wavefront correction system.

23. A microtome according to claim 15, characterised in that it includes moreover a posteriori localisation means along at least one axis of the position of the bubble by detecting the light of the bubble plasma.

24. A microtome according to claim 15, characterised in that the block of material is a cornea (6) of an eye (5), wherein the axis Y corresponds substantially to the optical axis of the eye.

25. A microtome according to claim 24 characterised in that it comprises an adaptation part (8) made of a material of optical index substantially equal to that of the cornea is arranged on and matches at least the front surface of the cornea, said part having an input face for the convergent beam so that said convergent beam runs through elements having substantially the same optical index, wherein the input face is lateral.

26. A microtome according to claim 25, characterised in that the input face of the adaptation part (8) is planar and is such as the axis L of the convergent beam is substantially perpendicular to said input face.

27. A microtome according to claim 25, characterised in that the adaptation part (8) compresses and deforms at least the cornea.

28. A microtome according to claim 26, characterised in that the adaptation part (8) compresses and deforms at least the cornea.

29. A microtome according to any of the claim 25, characterised in that the space between the input face of the adaptation part (8) and the focusing means (2) is, in whole or in part, filled with a fluid of index substantially equal to that of the adaptation part (8) or of the focusing means (2).

30. A microtome according to any of the claim 26, characterised in that the space between the input face of the adaptation part (8) and the focusing means (2) is, in whole or in part, filled with a fluid of index substantially equal to that of the adaptation part (8) or of the focusing means (2).

31. A microtome according to any of the claim 27, characterised in that the space between the input face of the adaptation part (8) and the focusing means (2) is, in whole or in part, filled with a fluid of index substantially equal to that of the adaptation part (8) or of the focusing means (2).

32. An adaptation part (8) for a microtome, characterised in that it is especially suitable for implementation in the microtome of any one of the claim 24 and in that it is made of plastic material of optical index substantially equal to that of the cornea, it is of single-use type, and that it is intended for being arranged on and matching at least the front surface of the cornea, it has an input face for a convergent part of a laser beam, wherein said input face is lateral.
